(19) United States
(12) Patent Application Publication
(10) Pub. No.: US 2023/0042900 A1
(43) Pub. Date: Feb. 9, 2023

(54) SMART AND COMPACT IMAGE CAPTURE DEVICES FOR IN VIVO IMAGING

(71) Applicant: Nash Young, Palo Alto, CA (US)

(72) Inventor: Nash Young, Palo Alto, CA (US)

(21) Appl. No.: 17/396,333

(22) Filed: Aug. 6, 2021

(51) Int. Cl. A61B 1/05; A61B 1/06; A61B 1/04; G06T 7/00

(52) U.S. Cl. CPC A61B 1/05; A61B 1/0684; A61B 1/041; G06T 7/0014; G06T 2207/20081

(57) ABSTRACT

A novel in-vivo image capture device for capsule endoscope and its method of operation are described. The device includes a wafer level camera module design, high sensitivity backside illumination pixel with high definition image output and LED's to provide illumination, which is synchronized with an image sensor strobe signal. A frame rate of the device can be adjusted based on an angular motion detection from a gyroscope sensor, in which a high frame rate mode is maintained during fast motion while a low frame rate is maintained during slow or no motion. The image capture device also includes machine learning based SOC for image processing, enhancement, and compression. The SOC can process and store zone average of images. The image capture device also includes a high density flash storage to store images in the device, thus no RF transmitter is needed, which make the system more convenient to use.

### TECHNICAL FIELD OF THE INVENTION

The described embodiments relate generally to an image capture device for in vivo imaging. More particularly, the described embodiments relate to a swallowable image capture device with compact size, high quality image sensor and camera, integrated flash drive, integrated gyroscope sensor, synchronized image sensor and LED, machine learning based image capture, process, storage, and diagnosis.

### BACKGROUND

Estimated 19 million people in the US may suffer from disease related to small intestine, including obscure bleeding, irritable bowel syndrome, Crohn's disease, chronic diarrhea, and cancer. Early studies showed that capsule endoscope effectively visualized the entire small bowel and demonstrated a 71% superior diagnostic yield when compared to push enterostomy according to clinical trials reviewed by the Food and Drug Administration.

Current capsule endoscope systems on the market generally encompass large pill size, lower image quality, limited battery life, and a complicated system. The existing system on the market transfers an image out through a radio frequency signal, which a patient must wear several cables connecting to RF transceiver to download the images during the entire course of the procedure. Image may be easily lost or corrupted due to a bad RF connection. In most cases, image is received in poor quality in order to reduce image size and save battery life, which could impact a diagnosis.

It is important to utilize advanced semiconductor packaging technology to shrink the size of capsule, and at the same time to utilize the technology development on cameras and image sensors to improve image quality, as well as utilize system-on-chip technology for device control and machine learning based image process. It is also important to keep the system simple such that a patient may potentially perform the procedure at home and take the capsule to doctor for diagnosis after procedure done.

### SUMMARY OF THE INVENTION

Embodiments of the systems, devices, methods, and apparatus described in the present disclosure are directed to image capture devices, one or more synchronized light emitting diode, one or more gyroscopes, one ore more batteries, a power management unit, a system on chip for device control, image capture, image process, a compact flash drive for image storage, an output interface pin for image readout.

In a first aspect, the present disclosure describes an image capture device. The image capture device may include a compact camera design. The compact camera may include an image sensor, which may include an array of high sensitivity pixels. The image sensor may capture monochromic or RGB images. The image sensor may further feature a configurable operating frame rate to get optimized image quality with less power consumption. The compact camera may further include a lens to focus light onto an image sensor. The lens may be a fixed focus lens or an auto-focus lens design with a capacity to focus <3 cm distance. The lens may have a wide-angle field of view.

In another aspect, the present disclosure describes an image capture device. The image capture device may include one or array of light emitted diode (LED), the LED may output wide spectral light, the LED may operate in pulsed mode and synchronized with the image capture device.

In another aspect, the image capture device may include one or more batteries to provide power to the system. The image capture device may include a power management unit (PMU) to provide different supply voltages for other devices.

The image capture device may include one or more gyroscope sensors, the gyroscope sensor will sense an angular motion of the image capture device. An angular motion signal will be fed back to system to adjust a frame rate of the image sensor. The image sensor may work in a high frame rate to make sure to critical images will be taken. The image sensor may work in a low frame rate mode if there is a slow or no motion of the image capture device for power saving.

The image capture device may include a system-on-chip (SOC) to control the image sensor and the camera to process, enhance, compress image, and save output images to a flash drive. Based on real time image analysis, the SOC may provide control of the image sensor and the LEDs to adjust exposure time control, auto-gain control, and auto white balance; to adjust the image sensor frame rate or the operating mode based on the angular motion information obtained from the gyroscope sensor. The SOC may also process zone average of an image and save a time-stamped image only if an image is different from a previous captured image. Machine learning algorithm may also be used to analyze captured images and to identify images with critical feature, such as incorporating time stamps on images for doctor diagnosis.

The image capture device may include one high performance and high capacity flash drive to store all images. The content of flash drive may be transfer out to a computer through a special designed USB cable or other special interfaces.

In another aspect, the image capture device may further include one or more self-driven motor to control the motion of device within body.

In addition to the exemplary aspects and embodiments described above, further aspects and embodiments will become apparent by reference to the drawings and by study of the following description.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 is a schematic of cross-section illustration of an imaging capture device.

FIG. 2 shows a cross-section view of an image sensor and LED's.

FIG. 3 shows a typical block diagram of image sensor design.

FIG. 4 shows a typical schematic of an active pixel design.

FIG. 5 shows an example pixel readout timing diagram.

FIG. 6A shows a pixel array readout timing synchronized with LED pulse.

FIG. 6B shows a schematic of an image sensor strobe signal, a power management unit, and synchronized LED's.

FIG. 7 shows a drawing of a tradition camera design and a compact wafer level camera module design.

FIG. 8. shows a block diagram of image capture device in accordance with an embodiment of the invention.

FIG. 9 is a block diagram presentation of a method of system operation according to an embodiment of the invention.

FIG. 10 is a block diagram presentation of a method of image operation mode based gyroscope sensor angular motion detection.

FIG. 11 is a drawing of image zone average and process.

### DETAILED DESCRIPTION

Reference will now be made in detail to representative embodiments illustrated in the accompanying drawings. It should be understood that the following description is not intended to limit the embodiments to one preferred embodiment. To the contrary, it is intended to cover alternatives, modifications, and equivalents as may be included within the spirit and scope of the described embodiments as defined by the appended claim. Although references are made to an imaging device to be used in an endoscope procedure, this is by no means limiting and a person with ordinary skills in the art may appreciate that a similar device in the invention may be used in other in-vivo imaging as well.

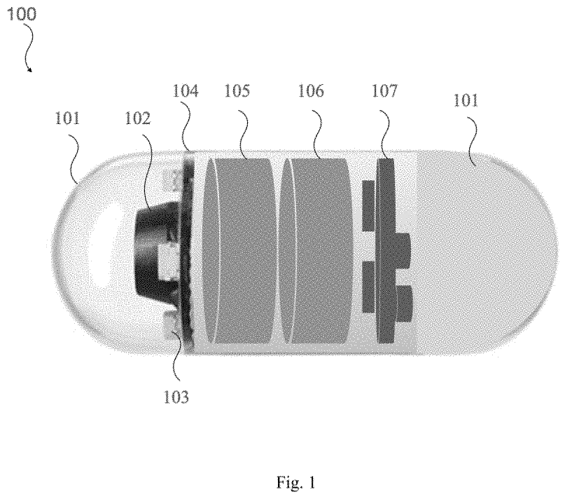

Reference is now made to FIG. 1, which illustrates an image capture device 100 and its components, according to one embodiment of the invention. The image capture device 100 in the invention typically comprises an optical window 101 and an imaging system 102 to capture an in-vivo image from a patient undergoing an endoscopy. The imaging system 102 comprises an illumination source 103, such as an all spectrum or white light emitting diode (LED). Both the imaging system 102 and the LED may be mounted on a printed circuit board 104. The printed circuit board 104 may be made of a flexible material. The image capture device 100 also includes one or more button batteries 105 and 106 to provide power to the image capture device during an endoscopic procedure. The imaging capture device 100 may also include one or more additional printed circuit board 107, which encompasses other necessary components for the image capture device 100, such as power management units, a computer chip for the device control and image processing, a non-volatile memory chip such as a flash memory to store all images, and interfaces to transfer images out from the flash memory. A gyroscope may also be included on the printed circuit board 107 for motion sensing and controlling of the imaging capture device 100. The printed circuit board 107 may also be made of a flexible material and it is electrically connected to the imaging system 102 so that the imaging system 102 is controlled by the components on the printed circuit board 107. A soft and transparent material provides not only a housing for the imaging system 102, the button batteries 105 and 106, the components on the printed circuit board 107, it but also forms the optical window 101.

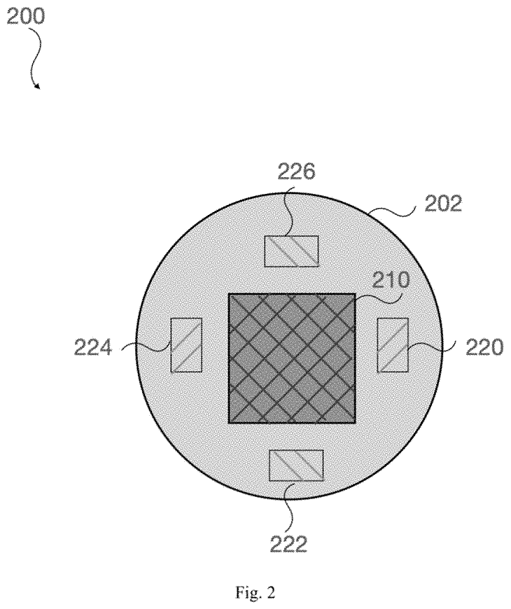

FIG. 2 illustrates the plan view of the imaging system 102, surrounded by LEDs 220, 222, 224, and 226. The number of LED's may be any odd or even number to provide adequate illumination to the imaging system 102. An image sensor 210 is typically located at center of the imaging system 102 with the LEDs on a periphery of the image sensor 210. The image sensor may be a charged-coupled device or more typically a backside illuminated image sensor made by CMOS technology. The LEDs support a pulsed operation mode which may be synchronized with an operation of the image sensor 210 to get optimal image quality with less power consumption.

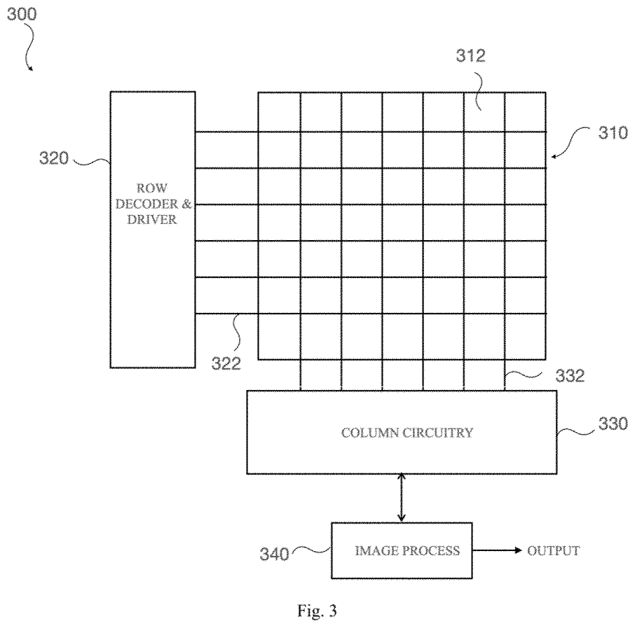

FIG. 3 is a block diagram of an example of an image sensor 300, which may be used in the image capture device as described with reference to FIG. 1. The image sensor 300 may include an image processor 340 and an imaging area 310. The imaging area 310 may be implemented as a pixel array that includes a plurality of pixels 312. The pixels 312 may be the same colored pixels (e.g., for a monochromatic imaging area 310) or differently colored pixels (e.g., for a multi-color imaging area 310). In the illustrated embodiment, the pixels 312 are arranged in rows and columns.

The imaging area 310 may be in communication with a column select circuit 330 through one or more column select lines 332, and with a row select circuit 320 through one or more row select lines 322. The row select circuit 320 may selectively activate a particular pixel 312 or group of pixels, such as all of the pixels 312 in a certain row. The column select circuit 330 may selectively receive the data output from a selected pixel 312 or group of pixels 312 (e.g., all of the pixels in a particular row). The row select circuit 320 and/or column select circuit 330 may be in communication with the image processor 340, which may process data from the pixels 312 and output that data to another processor, such as a system on a chip (SOC) included in on the printed circuit board 107.

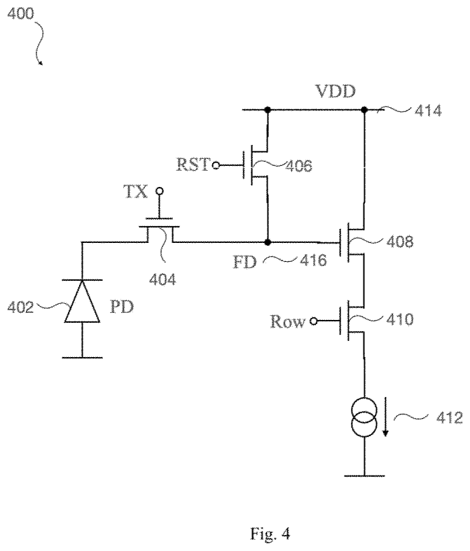

FIG. 4 shows an exemplary schematic design of a pixel 400. A photodetector 402 is used to convert the photo generated electron-hole (e-h) pairs into a photocurrent. A common photodetector 402 used in CMOS image sensors is the PIN photodiode, where a built-in p-n junction between a p-doped region and an n-doped region provides an electric field for the collection of generated charges by the photodetector. The actual number of e-h pairs generated by incident photons are measured by a quantum efficiency (QE) defined as the ratio of the photocurrent generated by the photodetector 402 to the photon flux incident on the photodetector 402. Quantum efficiency (QE) is one of the most critical parameters in pixel designs in a CMOS image sensor.

Besides the photodetector 402, the pixel 400 also comprises four transistors (4T) that include a transfer gate (TX) 404, a reset transistor (RST) 406, a source follower (SF) amplifier 408, and a row-select (Row) transistor 410. The transfer gate 404 separates the floating diffusion (FD) node 416 from the photodiode node 402, which makes the correlated double sampling (CDS) readout possible, and thus lower noise.

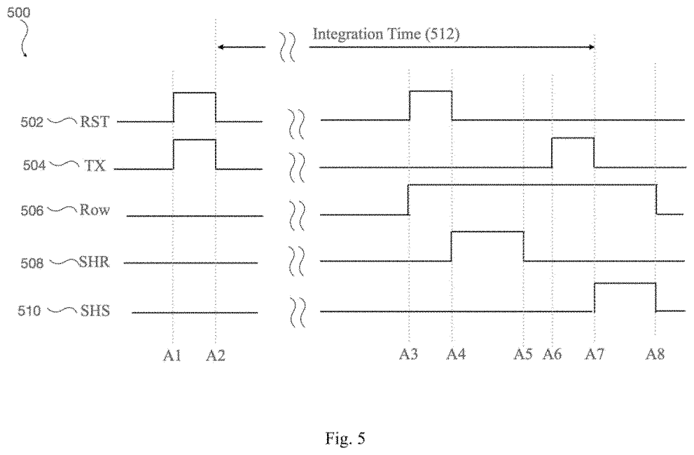

The readout timing diagram of a PIN photodiode is shown in FIG. 5. Prior to the integration time 512, both the TX gate 404 and the RST gate 406 are turned on at the same time at time A1, and a high voltage VDD 414 is applied to the floating diffusion node 416 and the photodetector 402 to fully deplete the p-n junction in the PIN photodiode. During the integration time 512, the photon-generated electrons are stored in the n+ region of the PIN photodiode and cause a lowering the potential of the n+ region of the p-n junction. During the pixel to column readout, the floating diffusion node 416 is first reset to VDD at time A3. A reset voltage may now be readout as SHR 508 (Sample and Hold RST) at time A4 for a true correlated double sampling. Next the transfer gate 404 is turned on at time A6, and the complete photon-generated electrons are transferred from the photodetector 402 to the floating diffusion node 416, which ensures a lag-free operation. Then the voltage is sampled again as SHS 510 for the true correlated double sampling and lower noise. A difference between SHR 508 and SHS 510 is corresponding to signal output level.

The integration time 512 is defined from a falling edge of TX gate 404 during the reset (time A2) to a falling edge of TX gate 404 during charge transfer (time A7). Normally the pixel response increases linearly with integration time 512 with a fixed amount light intensity.

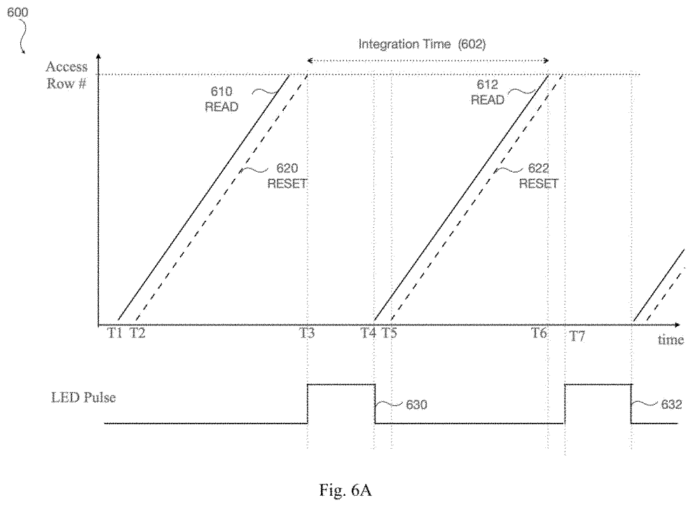

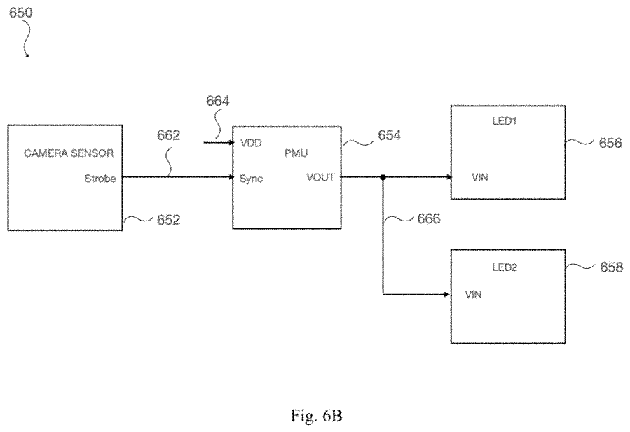

FIG. 6A illustrates a synchronized image sensor readout timing and LED strobe light pulsing. There are two operation modes for the LED, one is a constant wave mode and another is in a pulsed mode. It is preferred to operate LED in pulsed mode to reduce illumination during the time when the image sensor is not integrating thus reduce the power consumption during the endoscopy procedure. A row-by-row reset/readout operation is usually employed in a CMOS image sensor and the pixel-integration time 602 shifts by a row period between every row. This is a constraint for a strobe LED light timing. When the image sensor operates in a rolling reset mode, in order to keep all pixels exposed to the same amount of light, the LEDs are turned on only during the vertical blanking period. To further illustrate: first, the row reset scan is completed by time T2. The pixels on each row start integration after the reset scan 620. Then, the LEDs are turned on only during the integration time, i.e., from time T3 and T4 before the starting the first row pixels readout 610. In such way, the LEDs are turned off when during the readout time 610 and may operate in a power saving mode. The integration time 602 is exact the same for the whole image area and no dark shading issue with the image sensor.

The image sensor is designed to include one of a strobe control signal output pin 662 as shown in FIG. 6B. The strobe signal is synchronized with the image sensor readout vertical blanking period. The strobe signal will provide a sync signal input to PMU 654, which will generate a synchronized control signal 666 to turn all the LEDs, on and off in the pulsed mode, synchronized with the strobe signal from the image sensor. Even though the image sensor operates in an electronic rolling shutter mode, however, with the timing diagram as shown in FIG. 6A, the image sensor is working similarly as a global shutter sensor, which will save both power consumption for both the image sensor and the LEDs with less artifacts from motions of the image capture device during the endoscopy procedure.

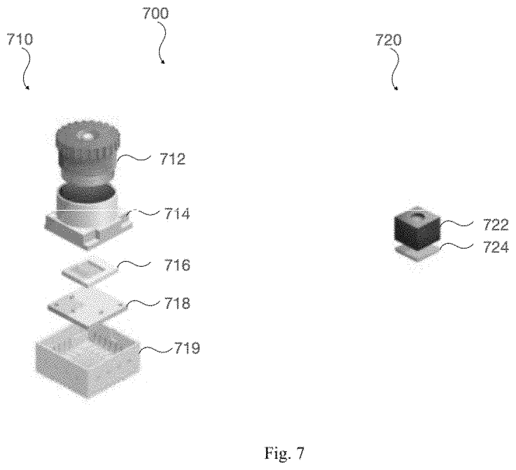

Now reference is made to FIG. 7, which illustrate a schematic drawing of a comparison between a traditional camera design 710 and a compact wafer level camera cube design 720. The traditional camera design 710 normally includes lens barrel 712, lens housing 714, image sensor 716, module substrate 718, module housing and pad connection 719. The lens barrel 712 focus light onto image sensor 716. Each component is separately made and an assembly is required to assemble the camera together. For in-vivo imaging such as capsule endoscopy, the lens is designed as a fix focus lens with a short focal length. It thus may be appreciated that it is quite a challenge to shrink the overall camera size, which is inevitably limiting the size of the image capture device.

Wafer-level optics and wafer-level chip-scale packaging technology may be used In order to make a compact camera system suitable for in-vivo endoscopy. In this invention, a highly integrated wafer level camera cube is proposed, which may include a lens focus element 722 and an image sensor 724. All the lens components may be manufactured using a wafer level processing and stacked in a wafer level using a wafer level chip scale packaging technology for the camera manufacturing process to integrate camera functionality in a small footprint and a low profile that fits in tiny space. The wafer level camera module design may be directly soldered to the printed circuit board 104 with no socket or insertion required.

For in-vivo imaging, a wafer-level integrated camera offers a few advantages compared with traditionally designed cameras. For example, the wafer-level integrated camera features a large field of view, e.g., a greater than 120-degree wide-angle lens design is preferred to capture as much light as possible such that critical information from an endoscopy-procedures will not missed from the image field of view or poor quality of the images. For another example, the camera lens 722 from a wafer-level integrated camera may focus on near focal distance with sharp focus, e.g., within 3 centimeter distance. The camera lens should have a low f-number and a large lens aperture to capture on more light onto image sensor to improve image quality at a low light situation.

For the purpose of capsule endoscopy and also this application, the image sensors must work in low light condition for most of time. Low light image quality is critical. Image sensor design choice should be carefully made to achieve optimal image quality with low power consumption, fast readout speed, and little image artifact or distortion. Since the camera lens design is circular and symmetric, it is preferred to design the image sensor with square pixel array to fully use the lens optical power, which means the pixel array has equal number of rows and columns to maximize light collect area. Square image sensors with 1280 rows and 1280 columns are recommended to get high-definition output either in x-direction or y-direction.

To achieve the optimal system performance, pixel size and pixel design have been carefully considered. A large pixel size will provide better low light performance but with higher cost due to a large die size and a larger footprint for the capsule image capture system. On the other hand, a smaller pixel will result in a smaller array size but the image quality suffers in low light conditions. Typically, a 1.0-1.4 μm pixel is good balance between the image quality and die size. A stacked chip back side illumination (BSI) image sensor is chosen over a front side illumination (FSI) image sensor for better low light performance. In addition, a back side illumination sensor provides many benefits over traditional front side illumination image sensor, such as a higher quantum efficiency (QE), lower cross-talks between pixels, a wide pixel acceptance angle, a less signal roll-off from array center to edge, thus is ideal for this application. Micro-lens design and optical stack may be fully optimized to achieve a higher QE, a lower cross-talk, and a less image flare or other artifacts.

Wafer bonding technology may be used to stack a logic wafer below a pixel wafer such that die size may be reduced significantly from traditional front side illumination image sensors. The logic wafer and the pixel wafer may be bonded in wafer level and connections between the wafers may be made through Cu—Cu hybrid bonding or TSV (Through Si Vias). Another benefit of stacked wafer technology is to use different technology node for the pixel wafer and the logic wafer. The pixel wafer may be made separately for the optimal pixel performance, while a more advanced process node may be adopted for the logic wafer to increase readout speed, reduce die size, add extra features, lower power consumption, and reduce cost. In addition, a memory wafer made of a dynamic random access memory or a NAND flash memory may also be attached by direct or hybrid wafer bonding to the logic wafer for an image storage and local processing of the images.

To improve image quality at low light, a readout noise from the image sensor must be reduced as much as possible. Correlated double sampling readout may remove kTC noise from RST gate 406 and reduce the readout noise by at least an order of magnitude. A low noise circuit design is also required for the pixel source follower amplifier 408, the pixel bias circuit 412, and a column amplifier and comparator circuitry of analog to digital converters (ADC).

A linear full-well capacity of the pixels defines the maximum signal to noise ratio of the image sensor and the sensor dynamic range. A typical linear full-well capacity is in a range of 6000e⁻ to 10000e⁻ for 1.0-1.4 μm pixel size, which provides an about 69-74 dB dynamic range, assuming a 2e⁻ readout noise. Other pixel parameters also need to be fully optimized to achieve the best possible image quality with a minimum power consumption.

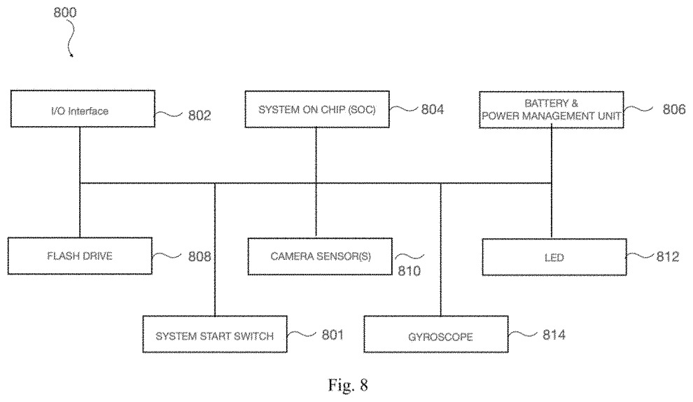

Now reference is made to FIG. 8, which is a block diagram of the image capture device 100 as also shown in FIG. 1. The image capture device 800 include a system start switch 801, one or more wide angle cameras 810, one or more white LEDs 812 to provide illumination to image capture device 800, a system on chip (SOC) 804 to control the device and image processing, one or more button batteries to provide power the image capture device 800 for 8-10 hours, a power management unit 806 to provide different power supplies to different components in the image capture device 800, a gyroscope 814 to track a motion of the image capture device 800 and provide a velocity information to SOC 804 to adjust a capture speed of the camera 810. A fast reading and storage flash drive 808 may also be included in the image capture device 800 to save time-stamped images. An I/O interface 802 is attached to the image capture device 800 to transfer the images from flash drive 808 to a computer.

The system start switch 801 may be controlled by an external magnet, which keeps the switch closed while it is in proximity to the switch. When a storage box is opened and the external magnet is moved away from, the system start switch 801 will turn on and to activate the SOC 804 and the camera 810 and image capture device 800 starts its operation. The camera 810 will capture images send them to the SOC 804 for processing, enhancement, and compression.

It is possible to integrate a high-speed large capacity flash drive 808 into the image capture device 800. The images taken from the endoscopic procedure may be stored in flash drive 808 with time stamps. A RF transmitter is not needed. At the end of the endoscopic procedure, an interface cable is used to transfer the images out from flash drive 808 for a diagnosis by a doctor.

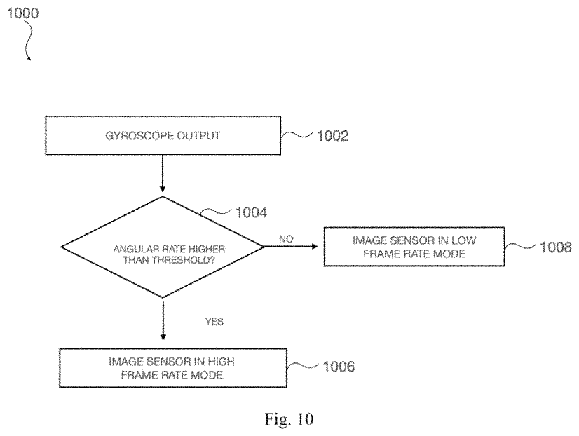

A gyroscope sensor 814 typically measures a rate of an angular motion of the image capture device 800, which is the rate of rotation. The gyroscope sensor, typically made of a microelectrical mechanical devices (MEMS) may measure three types of angular rate: yaw, pitch, and roll and the angular rate may be then converted into a linear velocity to detect the motion of the image capture device 800. The velocity of the image capture device 800, obtained from the gyroscope sensor 814, may be used to control the mode of operation of the image sensor 810. Reference is referring to FIG. 10 now. When the gyroscope sensor 814 detects an angular rate higher than a predefined threshold, which indicates a faster motion of the image capture device 800, as in situations when the image capture device 800 is moving through a wider tract in a patient's intestine, the camera 810 will operate in a high frame rate mode 1006, i.e, capture more images within a certain period of time, to avoid missing any critical features during the motion. When the angular rate of the image capture device 800 is below the predefined threshold, which indicates there is no or very litter motion from the image capture device 800, as in situations when the image capture device is moving through a very narrow intestine tract or stuck, the camera sensor 810 will operate in a low frame rate mode 1008 to avoid taking multiple images at the same area, in order to save battery power and save flash drive space for storage.

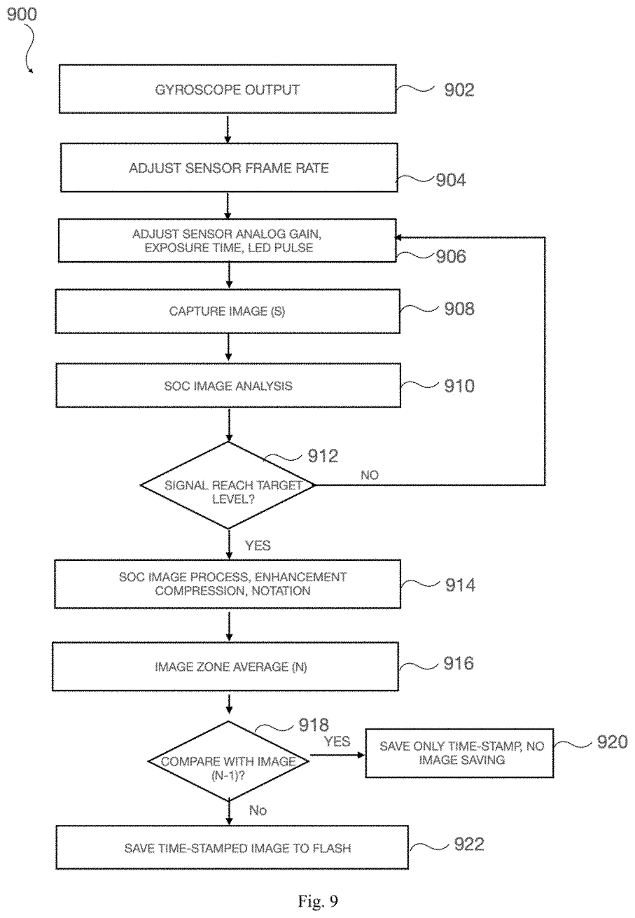

Now reference is made to FIG. 9, which is a block diagram of an operation of the image capture device 800. A gyroscope sensor detects the motion of the image capture device and the velocity of the image capture device is sent to a SOC and a frame rate of the camera is adjusted accordingly. In addition, a preliminary exposure time and a gain of the image sensor, the pulse of the LEDs will be set as accordingly. Image capture will start as shown with the new set of parameters and captured images are sent to the SOC or an on-chip image processor for processing. The image processor will check whether the images processed reach a certain auto-exposure target and will adjust the image sensor based on parameters such as an image sensor exposure, a LED pulse width, or an analog gain from the previously captured images, until the image output meet the auto-exposure target. Once the auto-exposure target is met, S(XC will further perform other processes on the images, such as a defect correction, a noise reduction, a sharpness enhancement, and an image format conversion, among others. Image compression is also part of the image processing, performed by the SOC, to reduce the image size while keep the original image quality as much as possible.

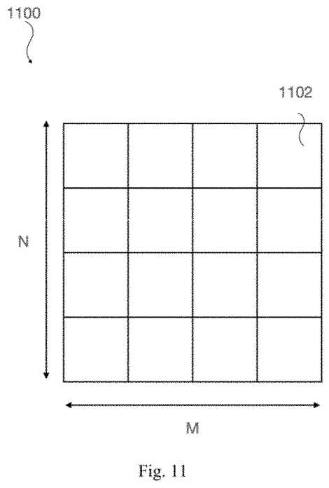

Reference is made to FIG. 11 now. A surface area of the image sensor as described previously may be allocated into zones, with each zone comprises one or more pixels, as shown in FIG. 11. Each image is separated into predefined M×N zones and a zone average for a particular image may be calculated and obtained by the on-chip image processor or the SOC. Output values of the pixels from within each zone 1102 may be averaged and saved in a SOC memory for further process. The zones may be equal in size, which comprises equal number of pixels; or different in size, in which each zone comprises different number of pixels, to improve accuracy. The zones may be adjacent to each or at least some of the zones may be overlapping. The average value of each zone may be compared with previous images. If difference of the zone averages from one or more zones between a current frame (N) and a previous frame (N−1) is below a certain threshold, which may indicate little or no extra information between the current frame (N) and the previous frame (N−1), and the current frame (N) can be discarded and there is no need to process further the current frame (N) in the SOC or save it into the flash drive. This typically happens when the image capture device is moving slowly or stuck in a patient's intestine track. This way, battery life and storage space of the flash drive may be saved. Only when the difference of zone averages from one or more zones between current frame (N) and previous frame (N−1) is higher than the threshold, the current frame (N) will be processed by the SOC and saved to the flash drive with a new time-stamp.

Once the system capture is done, the image capture device will be collected and sent to a doctor office for image transfer and analysis. The doctor office may have special devices to connect to the I/O pins inside the image capture device to transfer the time-stamped images for analysis. Machine-learning-based algorithm may be run to identify the images with associated with high risk areas for the doctors to focus on and narrow down the locations of interest. This may reduce the diagnosis time and increase the diagnosis efficiency.

What is claimed is:

1. A method of synchronizing an in vivo image capture device, comprising:
providing an image sensor, whereby the imaging sensor comprises plurality rows of pixels R1 to Rn, with n being an integral number;
integrating the pixels row by row;
reading the pixels row by row;
setting an integration time between the resetting of the last row of pixels Rn and the reading of the first row of pixels R1;
illuminating during the integration time;
processing readouts from the plurality rows of pixels in a control unit within the image capture device; and
transferring an image from the image capture device;
wherein the illuminating comprises providing a strobe signal from the image sensor and synchronizing the illuminating with the strobe signal.

2. The method in claim 1, wherein the illuminating comprises setting a pulse width of a LED.

3. The method in claim 1, further comprising detecting a velocity of the image capture device.

4. The method in claim 3, further comprising setting the integration time in proportionate to the velocity.

5. The method in claim 4, wherein the detecting comprising providing a gyroscope for detecting the velocity of the image capture device.

6. The method in claim 5, comprising:
setting an exposure time and a gain of the image sensor;
setting the pulse width of the LED;
obtaining the velocity of the image capture device; and
adjusting the exposure time and the gain of the image sensor according to the velocity; and
adjusting the pulse width of the LED according to the velocity.

7. The method in claim 1, wherein the transferring of the images comprises transmitting the images by a radio frequency transmitter.

8. The method in claim 1, further comprising storing the image in a memory storage unit.

9. The method in claim 8, wherein the storing the image comprises providing a non-volatile memory.

10. The method in claim 1, wherein the illuminating is performed only during the integration time.

11. An in vivo image capture device, comprising:
a housing;
an optical window and an optical system separated from the optical window;
a CMOS image sensor;
a LED;
a gyroscope;
a system start switch;
a battery;
a power management unit; and
a storage device.

12. The image capture device of claim 11, wherein the CMOS image sensor comprising an imaging area comprising an array of pixels, each pixel comprising a photodetector, pixel readout transistor, correlated double sampling readout; row select circuitry to select one or group of rows; column select circuitry to output one or group of column; one or more analog to digital converter to convert pixel output to digital output; output interface to output digital signal to other chips;

13. The image capture device of claim 12, wherein the CMOS image sensor has configurable register settings to change an integration time.

14. The image capture device of claim 12, wherein the CMOS image sensor has a strobe control signal to synchronize a vertical blank readout period with other devices in the image capture device.

15. The image capture device of claim 11, wherein the image capture device comprises a wide-angle lens; integrated wafer-level optics; and a camera made by wafer level chip scale packaging.

16. A method of operating an in vivo image capture device, comprising:
providing a CMOS image sensor having plurality of pixels;
allocating an imaging area of the CMOS image sensor into one or more zones, each zone having one or more pixels;
taking readouts from the pixels;
averaging readouts among neighboring pixels within the one or more zones;
comparing an average of readouts from a first frame to an average of readouts from a second frame within the one or more zones and determining a difference; and
processing the readouts from the plurality rows of pixels in the image capture device; and
transferring an image from the image capture device.

17. The method in claim 16, further comprising discarding the readouts from the second frame if the difference within the one or more zones is below a threshold value.

18. The method in claim 17, further comprising transferring the readouts from the first frame to a flash memory.

19. The method in claim 16, wherein the one or more zones are in equal size, having equal number of pixels.

20. The method in claim 16, wherein the one or more zones are overlapping.
